(19) United States
(12) Patent Application Publication
(10) Pub. No.: US 2005/0165486 A1
(43) Pub. Date: Jul. 28, 2005

(54) Prosthetic device and method

(75) Inventor: Hai H. Trieu, Cordova, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 10/830,247

(22) Filed: Apr. 22, 2004

Related U.S. Application Data
(63) Continuation-in-part of application No. 10/765,260, filed on Jan. 27, 2004.

(51) Int. Cl. A61F 2/44; A61F 2/30

(52) U.S. Cl. 623/17.13; 623/17.15; 623/23.4

(57) ABSTRACT

A prosthetic device and method, according to which the device includes a first and a second component, and at least one construct connected to the components for resisting at least one predetermined type of relative directional motion between the first and second components, and for allowing at least one other predetermined type of relative directional motion between the first and second components.

### BACKGROUND

The present disclosure relates generally to prosthetic devices and in particular to prosthetic devices that provide spinal stabilization.

In a human spine, intervertebral or spinal discs are located between the endplates of adjacent vertebrae (or adjacent backbones) to distribute forces between the vertebrae and cushion vertebral bodies. In some cases, spinal discs may rupture or degenerate to such a degree that surgical correction is required.

Typically, the surgical correction includes the removal of the spinal disc, and, in order to preserve the intervertebral disc space for proper spinal-column function, a prosthetic device is sometimes inserted between the adjacent vertebrae. In this context, prosthetic devices may be referred to as intervertebral prosthetic joints, prosthetic implants, disc prostheses or artificial discs, among other labels.

While preserving the intervertebral disc space for proper spinal-column function, most prosthetic devices allow at least one of the adjacent vertebrae to undergo different types of motion relative to the other, including bending and rotation. Bending may occur in several directions: flexion or forward bending, extension or backward bending, left-side bending (bending towards the human's left side), right-side bending (bending towards the human's right side), or any combination thereof. Rotation may occur in different directions: left rotation, that is, rotating towards the human's left side with the spinal column serving generally as an imaginary axis of rotation; and right rotation, that is, rotating towards the human's right side with the spinal column again serving generally as an imaginary axis of rotation.

In addition to the aforementioned motion types, some prosthetic devices further allow relative translation between the adjacent vertebrae in the anterior-posterior (front-to-back), posterior-anterior (back-to-front), medial-lateral right (middle-to-right side), or medial-lateral left (middle-to-left side) directions, or any combination thereof. Also, prosthetic devices may allow combinations of the different types of motion.

However, typical prosthetic devices are sometimes inadequate with respect to stabilizing the spine. For example, ligaments and other natural or artificial structures may be connected to adjacent vertebrae, and/or may extend between, from and/or along the adjacent vertebrae. These structures may serve to resist, restrict or prevent various relative directional motion types, limiting the allowable range of a particular type of motion in a particular direction and thereby stabilizing the spine. If, for any reason, these structures are missing or inoperable, typical prosthetic devices are unable to compensate for the accompanying loss of stabilizing functionality. In the absence of limiting or stabilizing structure, most prosthetic devices allow relatively large ranges of motion of various types and in various directions, possibly causing spinal instability.

Therefore, what is needed is a prosthetic device and/or method that provides spinal stabilization.

### BRIEF DESCRIPTION OF THE DRAWINGS

FIG. 1a is a perspective view of a prosthetic device according to one embodiment, the device including a flexible construct and upper and lower components.

FIG. 1b is a section view of the device of FIG. 1a taken along line 1b-1b.

FIG. 1c is a view similar to that of FIG. 1b but depicting another operational mode of the device.

FIG. 1d is a view similar to that of FIG. 1c but depicting yet another operational mode of the device.

FIG. 2a is a perspective view of a prosthetic device according to another embodiment, the device including a flexible construct and upper and lower components.

FIG. 2b is a detailed section view of a portion of the device of FIG. 2a taken along line 2b-2b.

FIG. 2c is a view similar to that of FIG. 2a but depicting another operational mode of the device.

FIG. 2d is a view similar to that of FIG. 2c but depicting yet another operational mode of the device.

FIG. 3 is a section view of a prosthetic device similar to that of the embodiment of FIG. 1b but having another flexible construct connected to the device.

FIG. 4 is an elevational view of a prosthetic device similar to that of the embodiment of FIG. 1a but having another flexible construct connected to the device.

FIG. 5 is an elevational view of a prosthetic device similar to that of the embodiment of FIG. 1a but having a wider flexible construct.

FIG. 6 is an elevational view of a prosthetic device similar to that of the embodiment of FIG. 1a but having another flexible construct connected to the device.

FIG. 7 is an elevational view of a prosthetic device similar to that of the embodiment of FIG. 6 but having another flexible construct connected to the device.

FIG. 8 is an elevational view of a prosthetic device according to another embodiment.

FIG. 9 is an elevational view of a prosthetic device similar to that of the embodiment of FIG. 8 but having another flexible construct connected to the device.

FIG. 10a is a perspective view of a prosthetic device according to another embodiment.

FIG. 10b is a section view of the device of FIG. 10a taken along line 10b-10b.

FIG. 11a is a perspective view of a prosthetic device according to yet another embodiment.

FIG. 11b is a section view of the device of FIG. 11a taken along line 11b-11b.

FIG. 11c is a view similar to that of FIG. 11b but depicting another operational mode.

FIG. 12a is a perspective view of a prosthetic device according to yet another embodiment.

FIG. 12b is a section view of the device of FIG. 12a taken along line 12b-12b.

FIG. 13a is a perspective view of a prosthetic device according to yet another embodiment. 11 FIG. 13b is a section view of the device of FIG. 13a taken along line 13b-13b.

FIG. 13c is an elevational view of the device of FIG. 13a.

FIG. 14 is a view similar to that of a portion of FIG. 1b but depicting a spiked washer.

FIG. 15 is a view similar to that of FIG. 2b but depicting a spiked washer.

FIG. 16 is a view depicting another means for connecting a construct to a component.

FIGS. 17 through 20 are views, similar to that of FIG. 1b, but depicting alternative embodiments having alternative disc prostheses.

### DETAILED DESCRIPTION

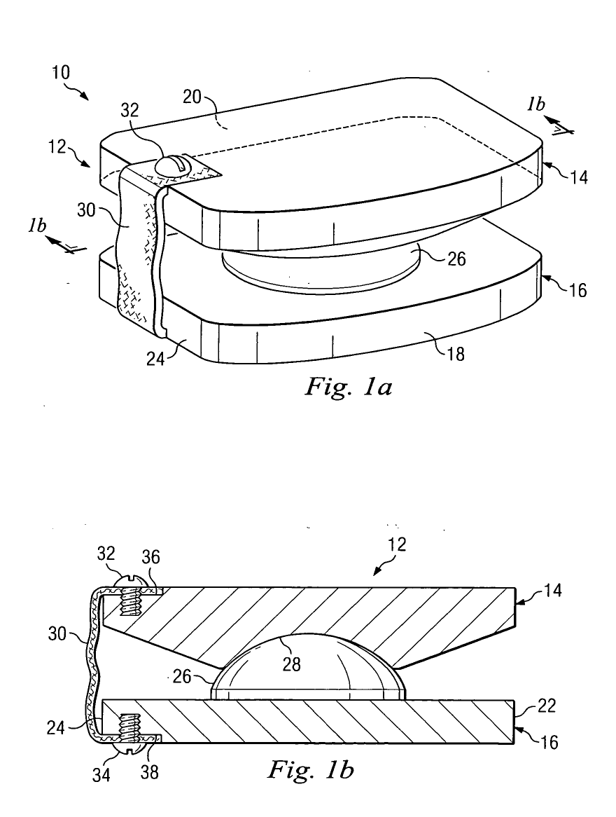

Referring to FIGS. 1a and 1b, a prosthetic device is generally referred to by the reference numeral 10. The device 10 includes a disc prosthesis or artificial disc 12 having an upper plate-like component 14 and a lower plate-like component 16. It is understood that the disc 12 is an articulating joint, configured for disposition within an intervertebral space between adjacent vertebral bodies in a human spine, that maintains or restores motion by providing relative bending and rotational motion between the vertebral bodies. The disc 12, along with the components 14 and 16, includes an anterior side 18, a posterior side 20, a left lateral side 22 and a right lateral side 24. A convex-shaped projection 26 extends from the component 16 and engages an articular surface defined by a concave recess 28 formed in the component 14. The disc 12 may include additional structure and other features not shown but disclosed in detail in other patents and/or patent publications such as, for example, U.S. Patent Publication No. 2003/0208273 (Ser. No. 10/042,589), the disclosure of which is incorporated by reference.

A flexible construct 30 extends vertically from the component 14 to the component 16 on the right lateral side 24, outside of the disc 12 and about the outer edges of the components. Threaded fasteners 32 and 34 connect the construct 30 to the components 14 and 16, respectively. As shown in FIG. 1b, recesses 36 and 38 are formed in the components 14 and 16, respectively, to receive the construct 30. The construct 30 is sized so that it is relaxed and not undergoing tension when the disc 12 is in its neutral position, as shown in FIG. 1b.

The components 14 and 16 may pivot and/or rotate relative to each other. In particular and referring to FIG. 1c, the device 10 may undergo left-side bending, that is, the component 14 may pivot towards the left lateral side 22 of the component 16 as shown by the arrow. After the component 14 pivots to a certain predetermined degree, the construct 30 is tensioned so that it begins to resist or restrict this bending. As the component 14 continues to pivot, the construct 30 continues to restrict or resist the left-side bending until the construct is tensioned to the point that it prevents any additional pivoting, as shown in FIG. 1c, thereby limiting the allowable range of left-side bending, and stabilizing the disc 12 and the spine engaged therewith.

Referring to FIG. 1d, the device 10 may undergo right-side bending, that is, the component 14 may pivot towards the right lateral side 24 of the component 16 as shown by the arrow. Unlike during left-side bending, the construct 30 provides no resistance or restriction, allowing the disc 12 to undergo maximum right-side bending, limited only by the physical design of the disc 12, including its components 14 and 16.

The device 10 may undergo forward bending or flexion, wherein the component 14 pivots towards the anterior side 18 of the component 16, or backward bending or extension, wherein the component 14 pivots towards the posterior side 20 of the component 16. The construct 30 does not resist, limit or restrict this flexion or extension.

Also, the device 10 may undergo left rotation wherein the component 14 rotates counterclockwise, or right rotation wherein the component 14 rotates clockwise. The construct 30 is sized to resist or restrict left and right rotation of the component 14 and ultimately to prevent predetermined excessive levels of left and right rotation of the component 14, thereby limiting the allowable range of left and right rotation, and stabilizing the disc 12 and the spine engaged therewith. It is understood that, under certain conditions, the component 16 may also experience the types of relative directional motion described above in connection with the component 14, moving relative to the component 14.

Due to the initial vertical extension of the construct 30, it is understood that left-side bending may be considered the primary motion restriction of interest whereas left and right rotation may be considered secondary motion restrictions of interest. Moreover, it is understood that the construct 30 may be sized so that it has no “slack” and therefore substantially prevents any left-side bending. Thus, it is understood that the degree of slack in the construct 30 at the neutral position controls the allowable range of motion in the selected direction, that is, the left-side bending range of motion.

Further, it is understood that different types of motion may be controlled in the device 10, that is, the construct 30 may be connected to the components 14 and 16 on the anterior side 18, the posterior side 20 or the left lateral side 22 in order to primarily restrict extension, flexion or right-side bending, respectively (while also continuing to restrict left and right rotation).

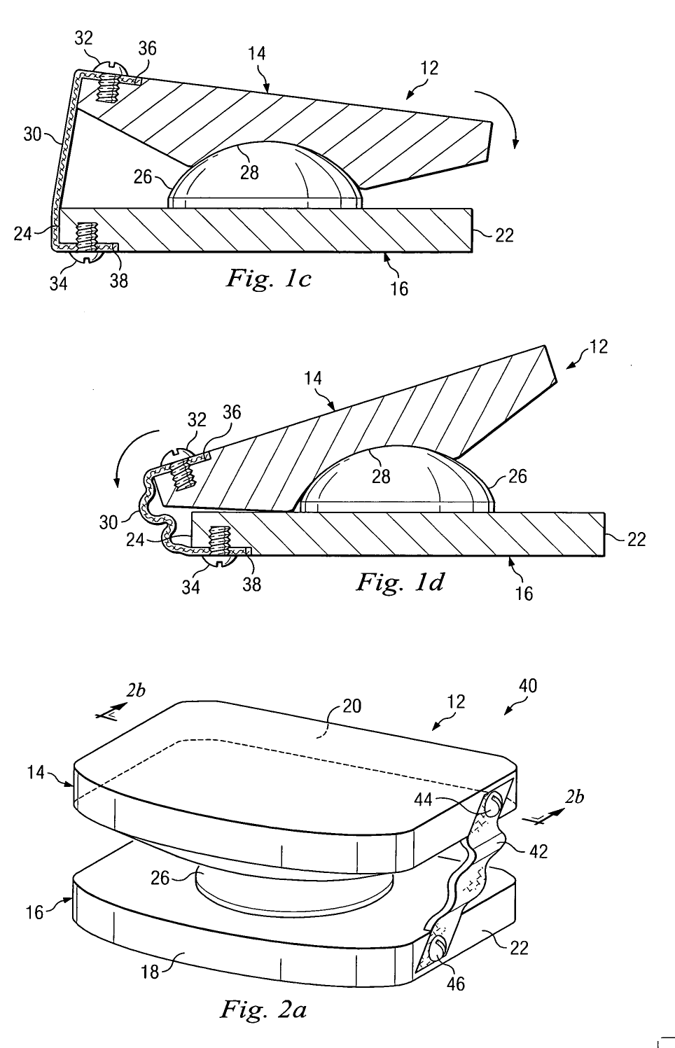

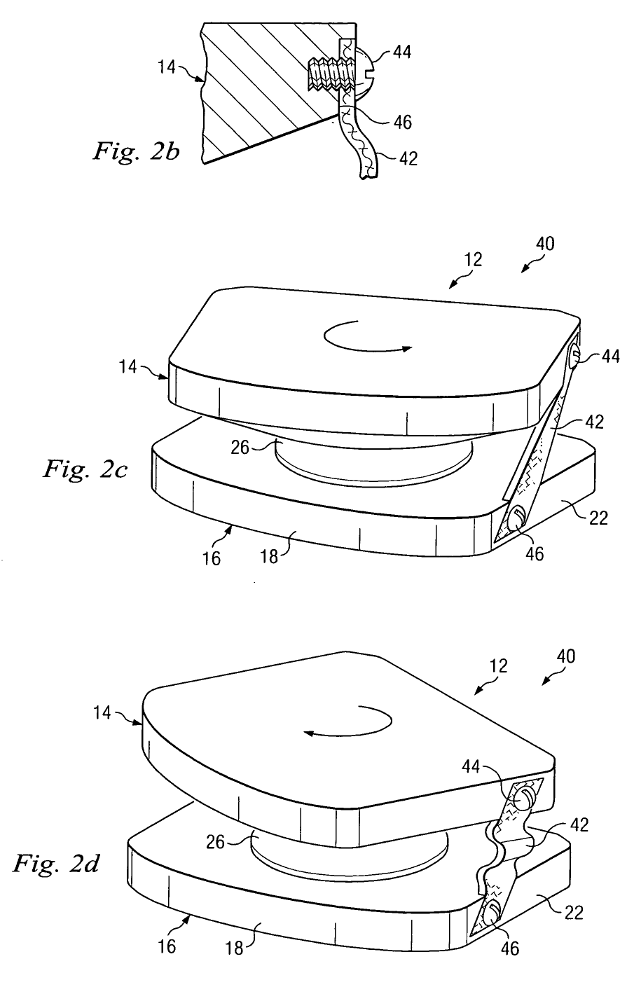

FIGS. 2a through 20 depict prosthetic devices according to alternative embodiments. It is understood that all of the artificial discs in these embodiments are configured for disposition within an intervertebral space between adjacent vertebral bodies in a human spine, and maintain or restore motion by providing relative motion between the vertebral bodies, as discussed above. Also, the terms “flexion,” “extension,” “left-side bending,” “right-side bending,” “left rotation,” “right rotation,” “medial-lateral right translation,” “medial-lateral left translation,” “anterior-posterior translation” and “posterior-anterior translation,” as discussed above, are applicable to one or more of the below-described embodiments.

Referring to FIGS. 2a and 2b, another embodiment of a prosthetic device is generally referred to by the reference numeral 40, and is similar to that of FIGS. 1a through 1d and contains several parts of the embodiment which are given the same reference numerals. In the embodiment of FIGS. 2a and 2b, a flexible construct 42 diagonally extends from the component 14 to the component 16 on the left lateral side 22.

Threaded fasteners 44 and 46 connect the construct 42 to the components 14 and 16, respectively, with the fastener 44 positioned towards the posterior side 20 and the fastener 46 positioned towards the anterior side 18. As shown in FIG. 2b, a recess 46 is formed in the component 14 to receive the construct 42. Although hidden from view in FIG. 2a, it is understood that a recess is also formed in the component 16 to receive the construct 42. The construct 42 is sized so that it is relaxed and not undergoing tension when the disc 12 is in its neutral position, as shown in FIG. 2a.

In operation, the components 14 and 16 may pivot and/or rotate relative to each other. Referring to FIG. 2c, the device 40 may undergo left rotation (or counterclockwise rotation). As shown by the arrow, the component 14 rotates counterclockwise relative to the component 16, and the construct 42 is tensioned so that it begins to restrict or resist the rotation. As the component 14 continues to rotate, the construct 30 continues to restrict or resist the rotation until the construct substantially prevents any additional rotation, as shown in FIG. 2c, thereby stabilizing the disc 12 and the spine engaged therewith.

Referring to FIG. 2d, the device 40 may undergo right rotation as shown by the arrow. Unlike during left rotation, the construct 42 provides no resistance, allowing the disc 12 to undergo maximum right rotation, limited only by the physical design of the disc 12, including its components 14 and 16. Similarly, the construct 42 does not restrict or resist flexion, extension, left-side bending or right-side bending.

Due to the initial diagonal extension of the construct 42, it is understood that left rotation is the primary motion restriction of interest for this embodiment. Moreover, it is understood that the construct 42 may be sized so that it substantially prevents any left rotation. Further, it is understood that the construct 42 may extend diagonally in a manner with the fastener 44 positioned towards the anterior side 18 and the fastener 46 positioned towards the posterior side 20, so that right rotation is restricted. Still further, it is understood that the construct 42 may extend from the component 14 to the component 16 on the anterior side 18, the posterior side 20 or the left lateral side 22.

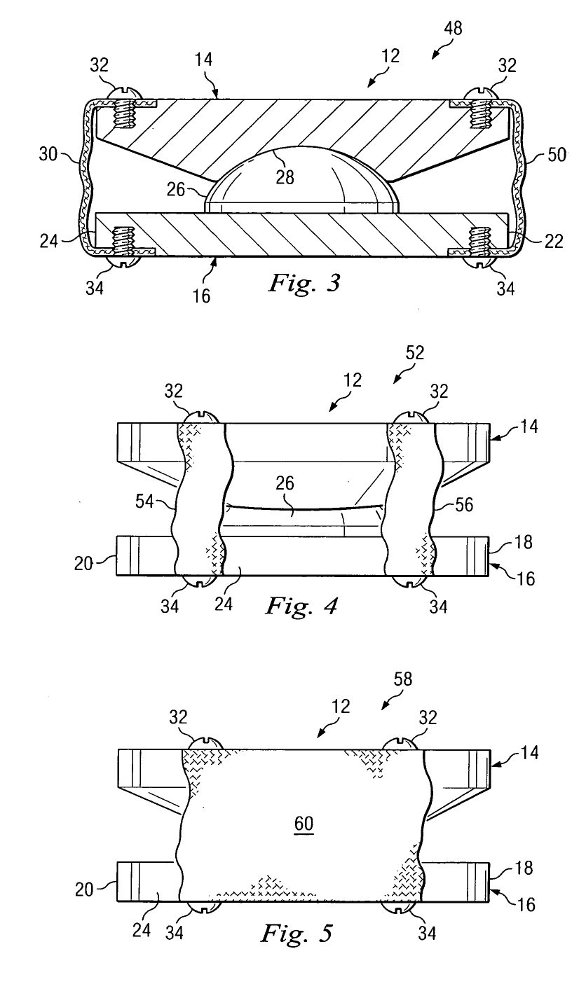

Referring to FIGS. 3 through 6, another embodiment of a prosthetic device is shown in each figure, and each embodiment contains several parts of the embodiment of FIGS. 1a through 1d which are given the same reference numerals. Each embodiment differs as to the quantity and configuration of the flexible construct or constructs.

In FIG. 3, a prosthetic device 48 includes an additional vertically-extending flexible construct 50 connected to components 14 and 16 on the left lateral side 22. In operation, left and right-side bending are resisted or restricted and/or prevented by the constructs 30 and 50. Also, left or right rotation is resisted or restricted and ultimately may be prevented by the constructs 30 and 50. Flexion and extension are not restricted or limited. It is understood that, instead of the left lateral side 22 and the right lateral side 24, the constructs 30 and 50 may be connected to the components 14 and 16 on the anterior side 18 and the posterior side 20, respectively, to primarily restrict and/or prevent flexion and extension and secondarily restrict left and right rotation, while allowing left-side and right-side bending.

As shown in FIG. 4, a prosthetic device 52 includes two vertically-extending flexible constructs 54 and 56 connected to the components 14 and 16 on the right lateral side 24. In operation, the constructs 54 and 56 primarily restrict and/or prevent left-side bending and secondarily restrict left and right rotation. Also, flexion or extension may be restricted while right-side bending is not restricted or limited. It is understood that if the constructs 54 and 56 are identical to the construct 30 of the embodiment of FIGS. 1a through 1d, then the constructs 54 and 56 provide approximately twice as much left-side-bending resistance as the construct 30.

FIG. 5 depicts a prosthetic device 58 including a vertically-extending flexible construct 60, connected to the components 14 and 16 on the right lateral side 24, that is wider than the construct 30 of the device 10. In operation, the construct 60 primarily resists and/or prevents left-side bending and secondarily restricts left and right rotation. Also, flexion and extension may be restricted due to the width of the construct 60. Right-side bending is not restricted or limited.

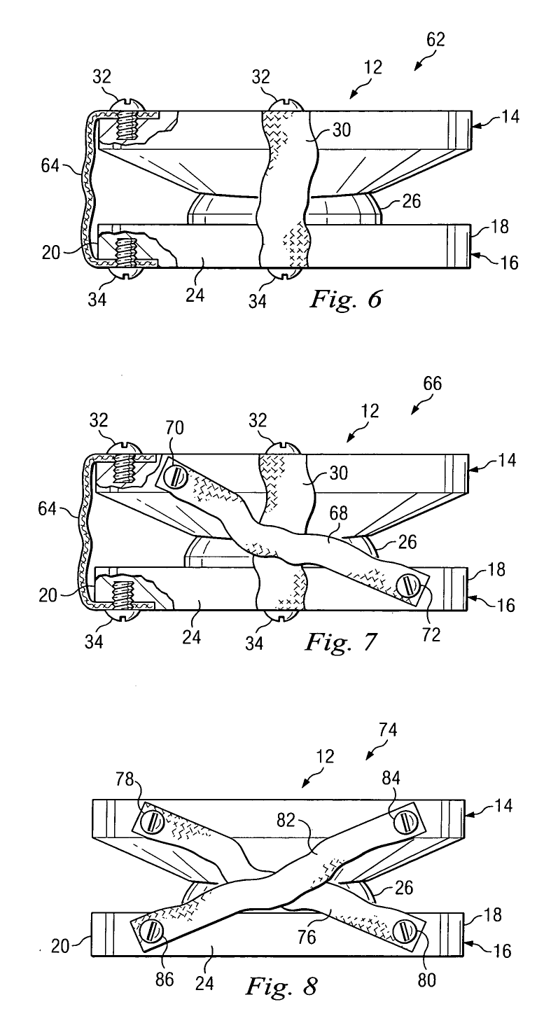

In FIG. 6, a prosthetic device 62 includes a vertically-extending flexible construct 64 connected to the components 14 and 16 on the posterior side 20. In operation, in addition to the above-described operation involving the construct 30, the construct 64 primarily restricts and/or prevents flexion and secondarily restricts left and right rotation. Extension and right-side bending are not restricted or limited.

Referring to FIG. 7, another embodiment of a prosthetic device is generally referred to by the reference numeral 66, and is similar to that of FIG. 6 and contains several parts of the embodiment which are given the same reference numerals. In the embodiment of FIG. 7, a flexible construct 68 diagonally extends from the component 14 to the component 16 on the right lateral side 24. Threaded fasteners 70 and 72 connect the construct 68 to the, components 14 and 16, respectively, with the fastener 70 positioned towards the posterior side 20 and the fastener 72 positioned towards the anterior side 18. In operation, in addition to the above-described operation involving the constructs 30 and 64, the construct 68 restricts and/or prevents right rotation. Extension and right-side bending are not restricted or limited.

Referring to FIG. 8, another embodiment of a prosthetic device is generally referred to by the reference numeral 74, and is similar to that of FIGS. 1a through 1d and contains several parts of the embodiment which are given the same reference numerals. In the embodiment of FIG. 8, a flexible construct 76 is connected to and diagonally extends from the component 14 to the component 16 on the right lateral side 24.

Threaded fasteners 78 and 80 connect the construct 76 to the components 14 and 16, respectively, with the fastener 78 positioned towards the posterior side 20 and the fastener 80 positioned towards the anterior side 18. Similarly, a flexible construct 82 is connected to and diagonally extends from the component 14 to the component 16 on the right lateral side 24. Threaded fasteners 84 and 86 connect the construct 82 to the components 14 and 16, respectively, with the fastener 84 positioned towards the anterior side 18 and the fastener 86 positioned towards the posterior side 20. In operation, the construct 76 restricts right rotation while the construct 82 restricts left rotation. Left-side bending, right-side bending, flexion and extension are not restricted or limited.

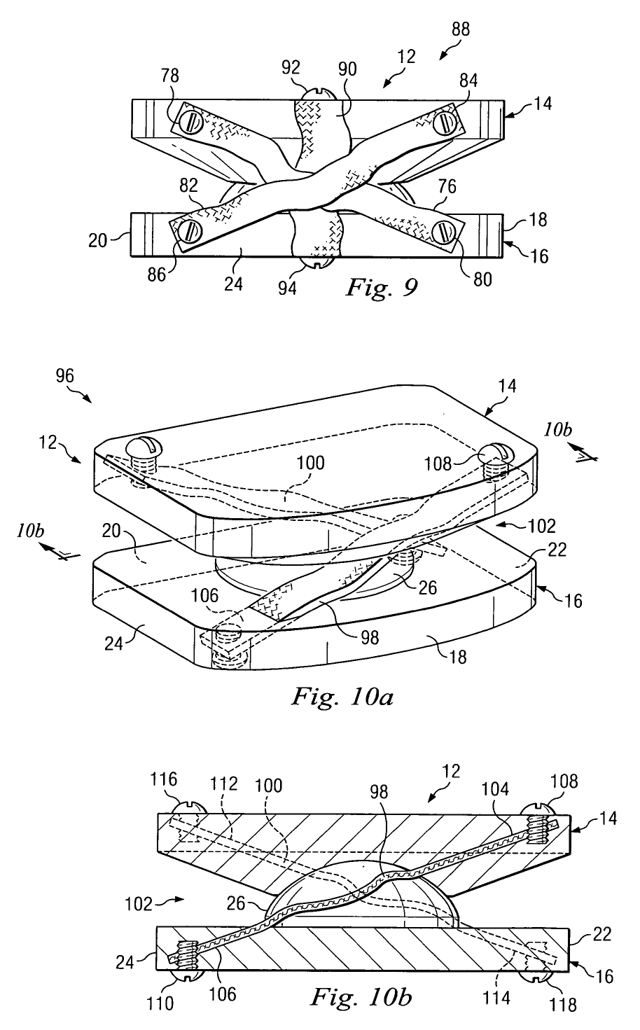

Referring to FIG. 9, another embodiment of a prosthetic device is generally referred to by the reference numeral 88, and is similar to that of FIG. 8 and contains several parts of the embodiment which are given the same reference numerals. In the embodiment of FIG. 9, a vertically-extending flexible construct 90 is connected to the components 14 and 16 on the right lateral side 24. Threaded fasteners 92 and 94 connect the construct 90 to the components 14 and 16, respectively. In operation, in addition to the above-described rotation restriction involving constructs 76 and 82, the construct 90 primarily restricts or resists left-side bending and secondarily resists left and right rotation. Right-side bending, flexion and extension are not restricted or limited.

For the embodiments of FIGS. 3 through 9, it is understood that additional flexible constructs may be installed on other sides of the disc 12, and that the number and relative positions of the flexible constructs may be varied to form an unlimited number of combinations or embodiments. These embodiments may be dictated by the various motions of interest which are to be resisted or restricted and/or substantially prevented, in order to stabilize the disc 12 and the spine engaged therewith. In addition, for the embodiments of FIGS. 7 through 9, the positions of the constructs may be varied with respect to their relative proximity to the center of the disc 12, that is, to the projection 26 of the disc 12. Any one of the constructs shown in these embodiments may be located outside of the other construct or constructs (farthest away from the projection 26), in between constructs, or on the inside of the other construct or constructs (nearest to the projection).

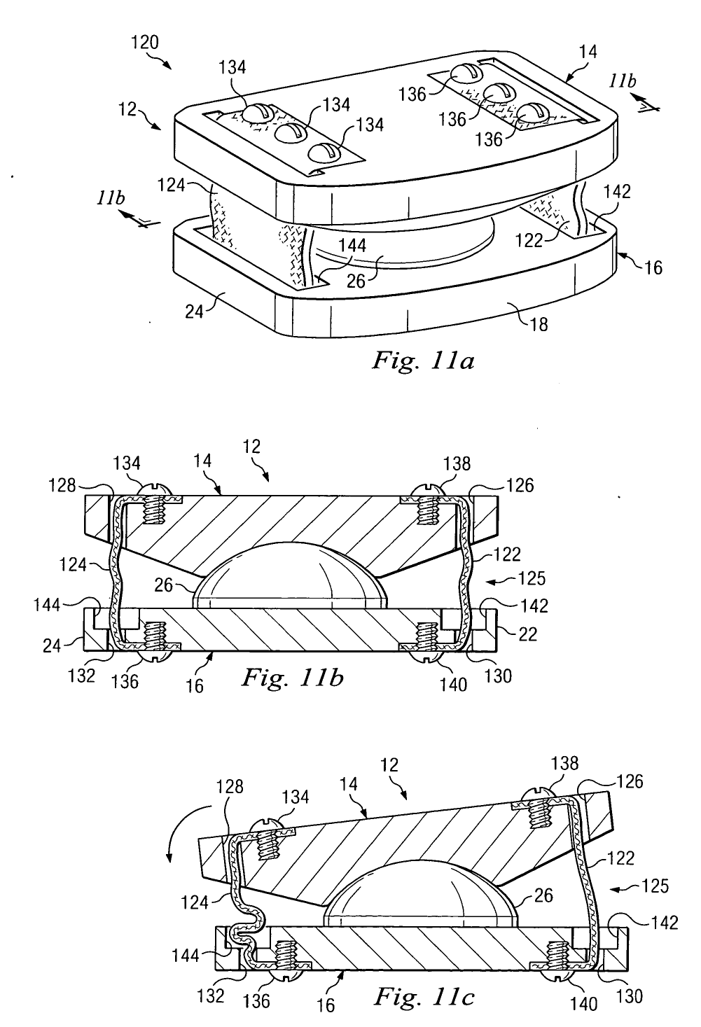

Referring to FIGS. 10a and 10b, another embodiment of a prosthetic device is generally referred to by the reference numeral 96, and is similar to that of FIGS. 1a through 1d and contains several parts of the embodiment which are given the same reference numerals. In the embodiment of FIG. 10, a pair flexible constructs 98 and 100 are connected to and diagonally extend between the components 14 and 16 in opposing directions on the anterior side 18 and the posterior side 20 of the projection 26, respectively. Unlike the embodiments of FIGS. 1a through 9 wherein the construct or constructs are positioned outside of the disc 12, the constructs 98 and 100 extend between the components 14 and 16 within a space 102 defined by the offset spacing of the components. The ends of the construct 98 are disposed through openings 104 and 106 formed in the components 14 and 16, respectively. Threaded fasteners 108 and 110 secure the construct 98 to the components 14 and 16, respectively. Similarly, the ends of the construct 100 are disposed through openings 112 and 114 formed in the components 14 and 16 and are secured thereto via threaded fasteners 116 and 118, respectively.

In operation, due to their opposing extension directions, the constructs 98 and 100 both resist or restrict and/or prevent left rotation. Right rotation, left-side bending, right-side bending, flexion and extension are not resisted, restricted or limited. It is understood that all of the constructs found in the embodiments of FIGS. 1a through 9, and all variations and combinations thereof, may be positioned between the components 14 and 16 and within a space defined by the spacing of the components in a manner similar to that of the embodiment of FIG. 10. Further, it is understood that constructs so positioned may be employed to cushion the component 14 and/or prevent at least a portion of the component 14 from contacting the component 16 when the component 14 is undergoing maximum bending in the direction where the construct is located, as allowed by the physical design of the disc 12.

Moreover, it is understood that channels may be formed in the component 16 corresponding to the relative locations of the constructs 98 and 100 to receive the constructs during either flexion or extension so as not to interfere with the bending motion, including the maximum bending motion allowed by the physical design of the disc 12. These types of channels will be described in more detail below.

Referring to FIGS. 11a and 11b, another embodiment of a prosthetic device is generally referred to by the reference numeral 120, and is similar to that of FIGS. 1a through 1d and contains several parts of the embodiment which are given the same reference numerals. In the embodiment of FIGS. 11a and 11b, a pair of opposing relatively wide flexible constructs 122 and 124 are connected to and vertically extend between the components 14 and 16 on the left lateral side 22 and the right lateral side 24 of the projection 26, respectively, in a space 125 defined by the offset spacing of the components. A pair of openings 126 and 128 are formed in the component 14 through which the constructs 122 and 124 are disposed, respectively. Likewise, a pair of openings 130 and 132 are formed in the component 16 through which the constructs 122 and 124 are disposed, respectively. Pluralities of threaded fasteners 134 and 136 connect the construct 122 to the components 14 and 16, respectively, and opposing pluralities of threaded fasteners 138 and 140 connect the construct 124 to the components 14 and 16, respectively. Channels 142 and 144 are formed in the component 16 and are sized and positioned so that they are relatively wider and generally coterminous with the openings 130 and 132, respectively, as shown in FIG. 11b.

In operation, the constructs 122 and 124 primarily restrict right and left-side bending, respectively, and secondarily restrict left and right rotation. Also, flexion and extension may be restricted or limited due to the widths of the constructs 122 and 124. During the allowable range of either left or right-side bending, the channel 142 or 144 receives the construct 122 or 124, respectively, so as not to interfere with the bending movement. As shown in FIG. 11c, the disc 12 undergoes right-side bending and the construct 124 is received by the channel 144 so as not to interfere with the bending motion. It is understood that similar channels may be formed in other embodiments wherein constructs are positioned between the components 14 and 16 so that the constructs in their relaxed states do not interfere with any type of desired relative motion between the components 14 and 16 of the disc 12.

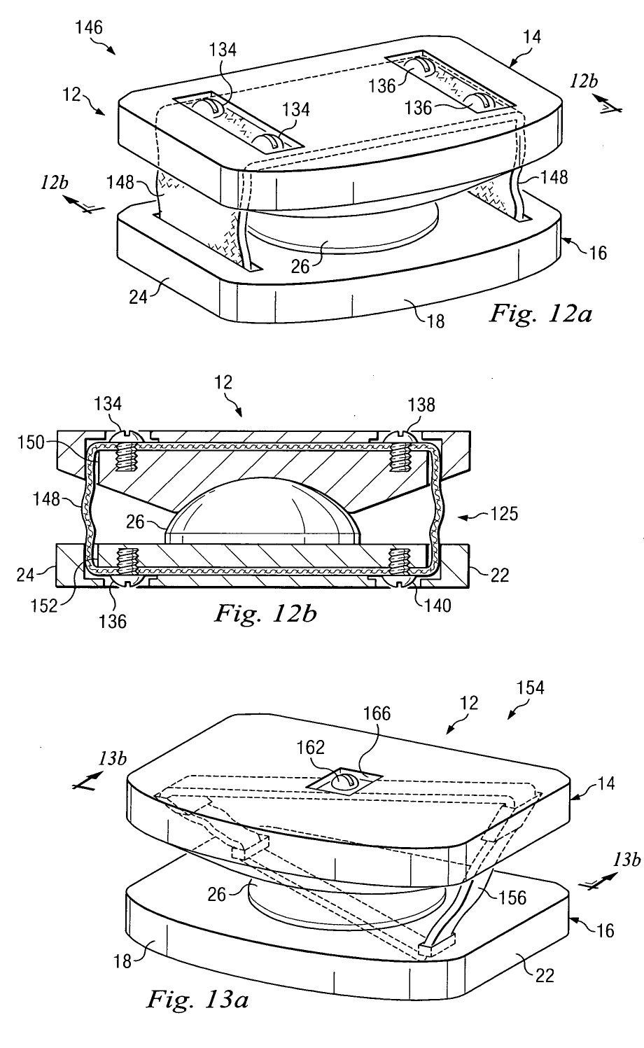

Referring to FIGS. 12a and 12b, another embodiment of a prosthetic device is generally referred to by the reference numeral 146, and is similar to that of FIGS. 11a through 11c and contains several parts of the embodiment which are given the same reference numerals. In the embodiment of FIGS. 12a and 12b, a single construct 148, in the form of a continuous loop, is connected to the components 14 and 16. U-shaped passages 150 and 152 are formed in the components 14 and 16, respectively. The construct 148 is disposed in the passages 150 and 152 so that the construct extends between the components 14 and 16 on either side of the projection 26. The operation of the embodiment of FIGS. 12a and 12b is similar to that of FIGS. 11a through 11c and will not be described. It is understood that the locations of the fasteners 134, 136, 138 and 140 may be varied in order to adjust the allowable ranges of motions in various directions.

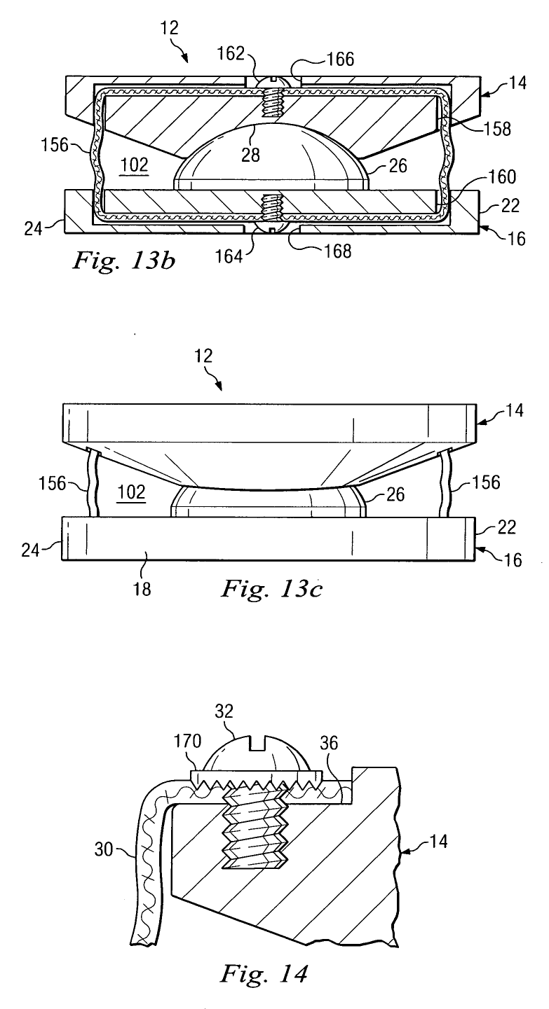

In FIGS. 13a and 13b, another embodiment of a prosthetic device is generally referred to by the reference numeral 154, and is similar to that of FIGS. 10a and 10b and contains several parts of the embodiment which are given the same reference numerals. In the embodiment of FIGS. 13a and 13b, a single continuous construct 156 is connected to the components 14 and 16. Passages 158 and 160 are formed in the components 14 and 16, respectively. The construct 156 is disposed in the passages 158 and 160 so that the construct extends diagonally between the components 14 and 16 on either side of the projection 26 in opposing directions, as shown in FIG. 13a. Threaded fasteners 162 and 164 connect the construct 156 to the components 14 and 16, respectively, via openings 166 and 168 providing access to the passages 158 and 160, respectively. The operation of the embodiment of FIGS. 13a and 13b is similar to that of FIGS. 10a and 10b and will not be described. It is understood that the locations of the fasteners 134 and 136 may be varied in order to adjust the allowable ranges of motions in various directions.

It is understood that the continuous-loop configurations of the constructs in the embodiments of FIGS. 12a through 13b may be applied to the above-described embodiments of prosthetic devices, and all variations and/or combinations thereof, wherein there are at two constructs extending from the component 14 to the component 16.

Based on the foregoing, it is readily appreciated that by strategically placing one or more flexible constructs on the outside of or between spaced components of a disc prosthesis, allowable ranges of motion in selected or preferred directions may be resisted, restricted, limited and/or substantially prevented, thereby stabilizing as desired the artificial disc and the spine engaged therewith.

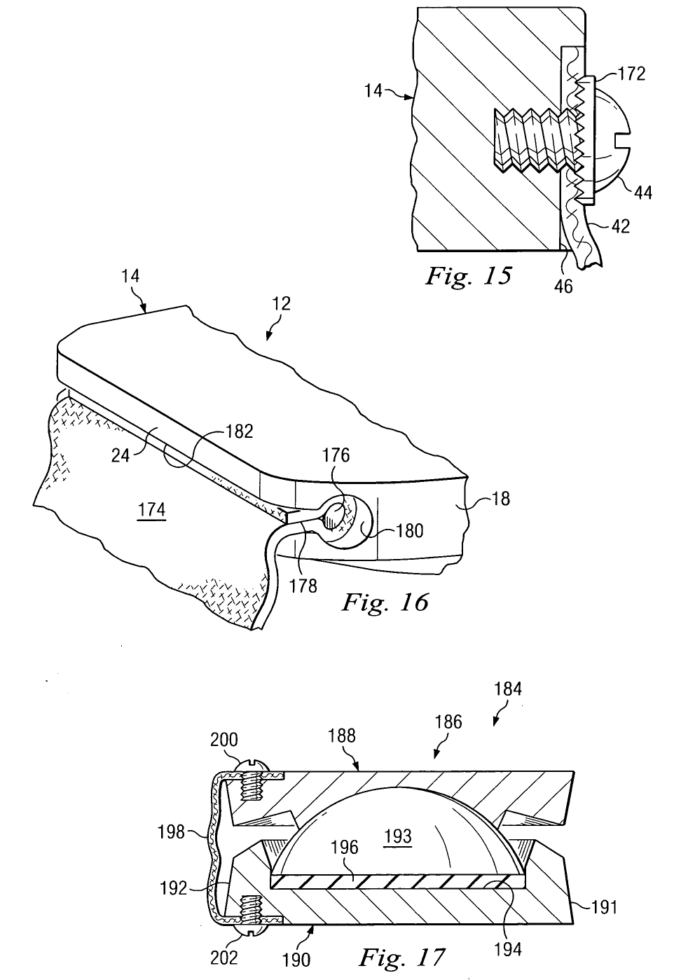

In addition to the above-described connections between the constructs and the components 14 and 16 in the embodiments found in FIGS. 1 through 13b, particularly in FIGS. 1b and 2b, and all variations and/or combinations thereof, it is understood that the above-described constructs may be connected to the components 14 and 16 using a wide variety of means. For example, as shown in FIG. 14, the embodiment is similar to that of FIG. 1b and contains several parts of the embodiment which are given the same reference numerals. However, in the embodiment of FIG. 14, a washer or plate 170 having spikes extending therefrom is disposed between the head of the fastener 32 and the construct 30, the spikes protruding into the construct to promote attachment. Likewise, the embodiment in FIG. 15 is similar to that of FIG. 2b and contains several parts of the embodiment which are given the same reference numerals. However, in the embodiment of FIG. 15, a washer or plate 172 having spikes extending therefrom is disposed between the head of the fastener 44 and the construct 42, the spikes protruding into the construct.

Referring to FIG. 16, the embodiment is similar to that of FIG. 1b and contains parts of the embodiment which are given the same reference numerals. In the embodiment of FIG. 16, the end of a flexible construct 174, having a width corresponding to the length of the right lateral side 24, is wrapped around a rod 176 and is sewn back onto itself, forming a seam 178. Openings 180 and 182 are formed in the component 14 towards the right lateral side 24 and extend the length of the side. The rod 176 is disposed in the opening 180 and the seam 178 is disposed in the opening 182. It is understood that the width of the construct 174, and the corresponding extension lengths of the openings 180 and 182, may be reduced.

In addition to the construct-component connection means found in the above-described embodiments, other means may be employed to connect the flexible constructs to the components 14 and 16 of the disc 12. The constructs may be fastened, fixed, anchored or locked to the components 14 and 16 using screws, pins, rivets, anchors, spiked washers or plates, knots, loops, adhesives or any combination thereof.

In addition to the above-described disc 12, it is understood that the prosthetic devices described above may be comprised of all types of disc prostheses or artificial discs, including articulating, non-articulating, elastic articulating, elastic or flexible disc designs. Although the disc 12 is an example of an articulating disc, other types of articulating discs may be used.

For example, referring to FIG. 17, a prosthetic device is generally referred to by the reference numeral 184. The device 184 includes an artificial disc 186 having a pair of components or endplates 188 and 190, a left lateral side 191 and a right lateral side 192. A hemispherical articulating component 193 is disposed in a cavity 194 formed in the endplate 190 and a washer 196 is interposed therebeteween. The disc 186 may include additional structure and other features not shown, and is similar to the PRODISC® modular implant and, among other embodiments, to prosthesis embodiments disclosed in U.S. Pat. No. 5,314,477 to Marnay, the disclosure of which is incorporated by reference.

A flexible construct 198 extends vertically from the endplate 188 to the endplate 190 on the right lateral side 192, outside of the disc 186 and about the outer edges of the endplates. Threaded fasteners 200 and 202 connect the construct 198 to the endplates 188 and 190, respectively. The construct 198 is sized so that it is relaxed and not undergoing tension when the disc 186 is in its neutral position as shown. The operation of the embodiment of FIG. 17 is similar to that of the embodiment of FIGS. 1a through 1d and will not be described.

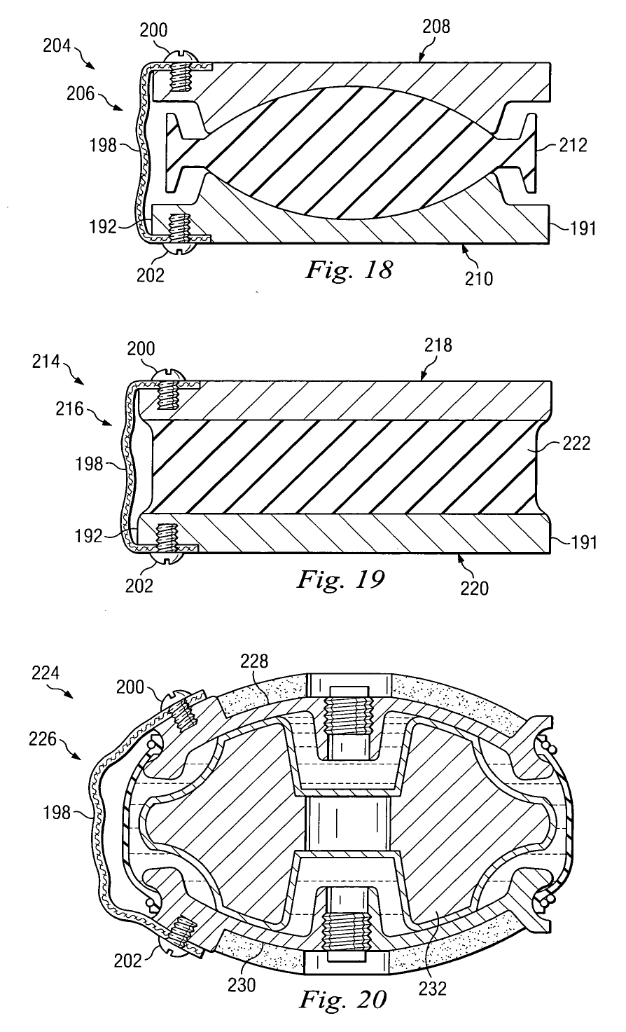

Referring to FIG. 18, another embodiment of a prosthetic device is generally referred to by the reference numeral 204, and is similar to that of FIG. 17 and contains some parts of the embodiment which are given the same reference numerals. In the embodiment of FIG. 18, an artificial disc 206 includes a pair of components or endplates 208 and 210, and a biconvex articulating component 212 disposed between the endplates. The construct 198 is connected to the endplates 208 and 210 via the fasteners 200 and 202, respectively. The disc 206 may include additional structure and other features not shown, and is similar to the CHARITÉ® intervertebral disc prosthesis and, among other embodiments, to prosthesis embodiments disclosed in U.S. Pat. No. 5,401,269 to Büttner-Janz et al., the disclosure of which is incorporated by reference.

In operation, the construct 198 operates in a manner similar to that of the embodiment of FIGS. 1a through 1d, at least with respect to any endplate 208 motion relative to the endplate 210—such motion may be considered to correspond to any component 14 motion relative to the component 16 of FIGS. 1a through 1d. In the embodiment of FIG. 18, the construct 198 may also restrict or resist and/or prevent the bending of the endplate 210 towards the left lateral side 191 of the endplate 208, and left and right rotation of the endplate 210 relative to the endplate 208.

Referring to FIG. 19, another embodiment of a prosthetic device is generally referred to by the reference numeral 214, and is similar to that of FIG. 17 and contains some parts of the embodiment which are given the same reference numerals. In the embodiment of FIG. 19, an artificial disc 216 includes a pair of components or endplates 218 and 220, and an elastomeric core 222 disposed between the endplates. The construct 198 is connected to the endplates 218 and 220 via the fasteners 200 and 202, respectively. The disc 216 may include additional structure and other features not shown, and is similar to the ACROFLEX™ disc and, among other embodiments, to prosthetic embodiments disclosed in U.S. Pat. No. 6,592,624 to Fraser et al., the disclosure of which is incorporated by reference.

In operation, the construct 198 operates in a manner similar to that of the embodiment of FIGS. 1a through 1d, at least with respect to any endplate 218 bending motion or rotation relative to the endplate 220—such motion may be considered to correspond to any component 14 bending motion or rotation relative to the component 16 of FIGS. 1a through 1d. In the embodiment of FIG. 19, the construct 198 may also restrict or resist and ultimately substantially prevent the bending of the endplate 220 towards the left lateral side 191 of the endplate 218, and left and right rotation of the endplate 220 relative to the endplate 218. Moreover, the construct 198 may restrict or resist and ultimately substantially prevent translation of either the endplate 218 or 220 relative to the other endplate. This translation restriction may occur in any of the above-described translation directions, or any combination thereof, and may reduce any shear loading on the elastomeric core 222 or its interfaces with the endplates 218 and 220.

Referring to FIG. 20, another embodiment of a prosthetic device is generally referred to by the reference numeral 224, and is similar to that of FIG. 17 and contains some parts of the embodiment which are given the same reference numerals. In the embodiment of FIG. 20, an artificial disc 226 includes a pair of components or shells 228 and 230, and a central body 232 disposed between the shells. The construct 198 is connected to the shells 228 and 230 via the fasteners 200 and 202, respectively. The additional structure and other features shown are disclosed in U.S. Patent Publication No. 2003/0135277 (Ser. No. 10/303,569), the disclosure of which is incorporated by reference. The disc 226 is similar to the BRYAN® disc prosthesis. In operation, the construct 198 operates in a manner similar to that of the embodiment of FIG. 19 and will not be described.

It is understood that all of the variations and/or combinations discussed and/or noted in connection with FIGS. 1 through 16, including construct shape, size, position, extension direction, quantity, operation and attachment means, among others, may be applied to the embodiments of FIGS. 17 through 20.

### ADDITIONAL VARIATIONS

It is understood that a wide variety of flexible constructs may be employed in the embodiments found in FIGS. 1 through 20, and all variations and/or combinations thereof. For example, various sizes of flexible constructs may be used, including various widths, lengths and thicknesses, resulting in various construct stiffnesses, as dictated by restriction and stabilization goals.

Also, the structural design, the material and/or the manufacturing method of the flexible constructs may be varied considerably. Regarding structural designs, the flexible constructs may be in a wide variety of forms such as, for example, braided bands and other types of bands, tape, tubing, tethers, cables, cords, fabric, mesh, sheets, chains, or any combination thereof. Combination examples may include, but are not limited to, a braided cord in braided tubing, fiber-reinforced tape and collapsed braided tubing. Another possible combination may be braided tubing or braided banding embedded in another material such as a polymer to enable two-stage performance, that is, the construct would be rigid for one range of tension (first stage), and flexible for another range of tension (second stage).

The flexible constructs may be composed of, for example, fiber-derived materials or any other solid materials, non-resorbable or resorbable polymer or polymers, metal or any combination thereof. Polymer types may include, but are not limited to, polyethylene, polyester, polyaryletherketone, polyamide, polytetrafluoroethylene, polyurethane, silicone, copolymers of silicone and polyurethane with or without end-group modifications, hydrogels, polyolefin-based rubber, polyisobutylene, polyisoprene, neoprene, nitrile rubber and vulcanized rubber. Bioresorbable polymer types may include, but are not limited to, polyactides, polyglycolides and various other copolymers. Metal types may include, but are not limited to, stainless steel, titanium, titanium alloys, shape-memory alloys or any combination thereof.

The flexible constructs may be manufactured by, for example, braiding, weaving, molding, extruding, casting, drawing or any combination thereof, or by any other manufacturing methods generally known in the art for fabricating the above-described structural designs using the above-described materials, among others. The method of manufacturing may be chosen to promote restriction in a selected or preferred direction. For example, the braiding or weaving patterns of the flexible constructs may be varied to modify the stiffnesses of the flexible constructs and the provided levels of resistance to motions in various directions.

Moreover, it is understood that the components 14 and 16 in the embodiments of FIGS. 1 through 16, the endplates 188 and 190, 208 and 210, and 218 and 220 in the embodiments of FIGS. 17 through 19, respectively, and the shells 228 and 230 in the embodiment of FIG. 20 may be composed of, for example, Cobalt-Chrome (Co—Cr) alloys, stainless steel, titanium alloys, alumina, zirconia, polycrystalline diamond compact, pyrolytic carbon, polyetheretherketone (PEEK), carbon-reinforced PEEK, ultra-high molecular weight polyethylene (UHMWPE), cross-linked UHMWPE or any combination or variation thereof, or multiple combinations or variations thereof for different portions of the components, endplates or shells. Also, it is understood that the surfaces of the components, endplates or shells may include serrations, spikes, ridges or fins, be grit-blasted, or be coated with, for example, porous bead coating, porous mesh coating, organoapatite (OA) coating, osteogenic peptide coating, growth factor coating, recombinant human bone morphogenetic protein (rh-BMP) coating, or any combination or variation thereof.

Also, it is understood that the above-described embodiments may be installed between adjacent vertebrae using typical techniques such as, for example, anterior, anterolateral, lateral or posterior approaches.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. For example, the present invention is not limited to an intervertebral prosthetic device and/or method, but may find further application in other areas of the human body such as, for example, areas associated with the knee.

The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the claims appended hereto and their equivalents. In the claims, means-plus-function clauses are intended to cover the structures described herein as performing the recited function and not only structural equivalents, but also equivalent structures.

1. A prosthetic device comprising a first component, a second component, and at least one construct connected to the first and second components for resisting at least one predetermined type of relative directional motion between the first and second components, and for allowing at least one other predetermined type of relative directional motion between the first and second components.

2. The device of claim 1 wherein the at least one other predetermined type of relative directional motion between the first and second components is opposite in direction to the at least one predetermined type of relative directional motion between the first and second components.

3. The device of claim 1 wherein the at least one predetermined type of relative directional motion between the first and second components is selected from the group consisting of flexion, extension, left-side bending, right-side bending, left rotation, right rotation, medial-lateral right translation, medial-lateral left translation, anterior-posterior translation or posterior-anterior translation.

4. The device of claim 1 wherein the at least one other predetermined type of relative directional motion between the first and second components is selected from the group consisting of flexion, extension, left-side bending, right-side bending, left rotation, right rotation, medial-lateral right translation, medial-lateral left translation, anterior-posterior translation or posterior-anterior translation.

5. The device of claim 1 further comprising at least one other construct connected to the first and second components.

6. The device of claim 1 further comprising means for connecting the construct to each of the first and second components.

7. The device of claim 6 wherein the connecting means comprises a member disposed in an opening formed in the first or second component, the member connected to the construct.

8. The device of claim 7 wherein the member is in the form of a rod and wherein the construct is wrapped around the rod.

9. The device of claim 1 wherein the construct is operable to cushion contact between the components.

10. The device of claim 1 further comprising a channel formed in at least one of the components for receiving at least a portion of the construct.

11. The device of claim 1 wherein the construct forms a continuous loop.

12. The device of claim 11 wherein at least two portions of the construct extend from the first component to the second component.

13. The device of claim 1 further comprising a passage formed in at least one of the components wherein at least a portion of the construct is disposed in the passage.

14. The device of claim 1 wherein the components are spaced apart.

15. The device of claim 14 wherein the construct is disposed within the space between the components.

16. The device of claim 14 wherein the construct is disposed outside of the space between the components.

17. The device of claim 14 wherein at least a portion of the construct extends outside of the space between the components.

18. The device of claim 1 further comprising a biconvex articulating component disposed between the first and second components.

19. The device of claim 1 further comprising a hemispherical articulating component disposed between the first and second components.

20. The device of claim 1 further comprising a convex-shaped projection disposed between the first and second components.

21. The device of claim 1 further comprising an elastomeric core disposed between the first and second components.

22. The device of claim 1 further comprising a central body disposed between the first and second components.

23. A method of controlling motion in a prosthetic device comprising providing a resistance to at least one predetermined type of relative directional motion between two components of the device, and allowing at least one other predetermined type of relative directional motion between the two components.

24. The method of claim 23 further comprising engaging the prosthetic device with a plurality of vertebral bodies in a spine.

25. The method of claim 23 wherein the step of providing comprises connecting at least one construct to the two components.

26. The method of claim 23 wherein the at least one predetermined type of relative directional motion between the two components is selected from the group consisting of flexion, extension, left-side bending, right-side bending, left rotation, right rotation, medial-lateral right translation, medial-lateral left translation, anterior-posterior translation or posterior-anterior translation.

27. The method of claim 23 wherein the at least one other predetermined type of relative directional motion between the two components is selected from the group consisting of flexion, extension, left-side bending, right-side bending, left rotation, right rotation, medial-lateral right translation, medial-lateral left translation, anterior-posterior translation or posterior-anterior translation.

28. The method of claim 23 wherein the at least one other predetermined type of relative directional motion between the two components is opposite in direction to the at least one predetermined type of relative directional motion between the two components.

29. A kit comprising a prosthetic device, and at least one construct connectable to the device for resisting at least one predetermined type of relative directional motion between two components of the device, and for allowing at least one other predetermined type of relative directional motion between the two components.

30. The kit of claim 29 wherein the at least one predetermined type of relative directional motion between the two components is selected from the group consisting of flexion, extension, left-side bending, right-side bending, left rotation, right rotation, medial-lateral right translation, medial-lateral left translation, anterior-posterior translation or posterior-anterior translation.

31. The kit of claim 29 wherein the at least one other predetermined type of relative directional motion between the two components is selected from the group consisting of flexion, extension, left-side bending, right-side bending, left rotation, right rotation, medial-lateral right translation, medial-lateral left translation, anterior-posterior translation or posterior-anterior translation.

32. The kit of claim 29 wherein the at least one other predetermined type of relative directional motion between the two components is opposite in direction to the at least one predetermined type of relative directional motion between the two components.
